(19) United States
(12) Patent Application Publication
(10) Pub. No.: US 2024/0317656 A1
(43) Pub. Date: Sep. 26, 2024

(54) SINGLE-STEP CATALYTIC PROCESS FOR THE PRODUCTION OF ALKYLATED AROMATICS USING CO2

(71) Applicant: COUNCIL OF SCIENTIFIC AND INDUSTRIAL RESEARCH, New Delhi (IN)

(72) Inventors: Anup PRAKASH TATHOD, Dehradun (IN); Anjan RAY, Dehradun (IN); Nagabhatla VISWANADHAM, Dehradun (IN); Selvamani ARUMUGAM, VISWANADHAM (IN)

(21) Appl. No.: 18/613,733

(22) Filed: Mar. 22, 2024

(30) Foreign Application Priority Data
Mar. 22, 2023 (IN) 202311020220

(51) Int. Cl. C07C 2/72; B01J 23/652; B01J 37/04; B01J 37/08; C07C 15/04; C07C 15/06; C07C 15/08

(52) U.S. Cl. CPC C07C 2/72; B01J 23/6522; B01J 37/04; B01J 37/082; C07C 15/04; C07C 15/06; C07C 15/08; C07C 2521/12; C07C 2523/652

(57) ABSTRACT

Utilization of CO₂ for the alkylation of aromatic hydrocarbons is one of the green and sustainable routes for the production of valuable alkylated aromatics like xylenes. Aspects of the present invention deal with the development of single-step catalytic process for the production of alkylated aromatics using CO₂ as a carbon source and alkylation reagent and methylcyclohexane as a hydrogen atom donor as well as source of toluene. In presence of the metal functionalized zeolite catalyst, methylcyclohexane undergoes dehydrogenation to produce toluene and hydrogen; hydrogen reacts with CO₂ to form active alkylating species which triggers the alkylation of toluene. Additionally, a novel process is disclosed for the production of xylene-rich alkylated aromatics from methylcyclohexane and CO₂ using single multi-functional catalyst possessing dehydrogenation, hydrogenation and acid functionalities.

### CROSS-REFERENCE TO RELATED APPLICATIONS

This application claims the benefit of priority from Indian Patent Application No. 202311020220, filed Mar. 22, 2023, the content of which is hereby incorporated herein in its entirety for all purposes.

### FIELD OF THE INVENTION

The present invention relates to a single-step catalytic process for the production of alkylated aromatics using CO₂ as a carbon source & alkylation reagent and methylcyclohexane as a hydrogen atom donor as well as source of toluene. Particularly, present invention relates to the conversion of methylcyclohexane and CO₂ into xylene-rich alkylated aromatics over metal functionalized zeolite-based catalyst without using molecular hydrogen. The present invention can be coupled to a toluene hydrogenation unit either co-located or remote, for methylcyclohexane supply.

### BACKGROUND

Utilization of fossil fuels to fulfill ever-increasing energy demand results in unsustainable levels of carbon emissions. CO2 is one of the main greenhouse gases inducing global warming and has well-known consequences on the environment and on human health. In this context, capture and utilization of CO2 for the production of chemicals is emerging as an important approach to mitigate its impact on the environment. Utilization of CO2 as an alkylation reagent for the production of alkylated aromatics could be a promising route for CO2 mitigation. Conventionally, this approach involves hydrogenation of CO2 to form an active alkylating species which then acts as an alkylation reagent. However, this process requires valuable molecular H2 for CO2 hydrogenation under energy-intensive enhanced pressure and temperature conditions to form such alkylated species. In this context, application of a liquid organic hydrogen carrier as a H2-donor instead of molecular H2 for CO2 hydrogenation is an attractive avenue for better process economics, milder operating conditions and improved net greenhouse gas mitigation.

Utilization of CO2 as an alkylation reagent instead of conventional alkylation reagents like methanol, dimethylether, methylchloride, methylbromide, methylcarbonate, acetaldehyde, dimethoxyethane, acetone, olefins, dimethylsulfide, etc., for the production of alkylated aromatics can fulfill the criteria of a green and environmentally benign process. Recently, alkylation of toluene using CO2 and molecular H2 has been reported by Zuo et al. (2020). However, application of liquid organic hydrogen carrier as H2-donor instead of molecular H2 for hydrogenation of CO2 is desirable from a process severity and net GHG stand point. Further, the process can be made more atom-economical if the dehydrogenated product of the H2-donor could be utilized in-situ during the course of reaction. Otherwise, the H2-donor molecule may remain as a low-value side product in the system that creates additional separation requirement or process complexity. Screening of such chemical compounds which can act as H2-donor as well as a reactant (in its dehydrogenated form) suggests that methylcyclohexane is an attractive option. This can serve as a H2-donor and its dehydrogenated form (toluene) can be used as a reactant for the production of alkylated aromatics. To the best of our knowledge, prior art for the alkylation of toluene or any other aromatic hydrocarbons using CO2 and methylcyclohexane as H2-donor to produce alkylated aromatics is not reported.

Reference can be made to the Chinese patent CN110015940A, which discloses a catalytic method for dehydrogenation of methylcyclohexane (hexahydrotoluene) under a carbon dioxide atmosphere to produce toluene and methane. The catalyst comprises of carrier; any one from the group of γ-Al2O₃, TiO2, SiO2, Mordenite, HZSM-5, or active carbon and the active component Ni, auxiliary agent Pt or Ce or both. Toluene and methane were the major products obtained in the reaction. Selectivity for toluene was observed to be 99.45% whereas extent of alkylation of toluene was very low with 0.55% selectivity for alkylated aromatics.

Reference can be made to the Chinese patent CN110028375A, which discloses a method wherein a reverse water gas shift reaction couples with dehydrogenation of methylcyclohexane (hexahydrotoluene) to produce toluene and CO. The catalyst comprises of carrier; any one from the group of γ-Al2O3, TiO2, SiO2, Mordenite, HZSM-5, or active carbon and the active metals (metal=Pt, Ni, Cu, Ce, Fe or combination thereof). Toluene and CO were the major products obtained in the reaction. Selectivity for toluene was observed to be 96.85% whereas extent of alkylation of toluene was very low with 0.22% selectivity for alkylated aromatics.

The present invention deviates from and innovates beyond the processes disclosed in patent CN110015940A and CN110028375A in terms of catalyst composition, reaction conditions and types of reactions involved. The processes disclosed in patent CN110015940A and CN110028375A relate to catalytic methods wherein two types of reaction i.e. i) dehydrogenation of methylcyclohexane to produce toluene and hydrogen and ii) reduction of CO2 to produce CH4 or CO takes place. In both processes extent of alkylation of toluene was negligible. In the present invention, a novel catalyst has been developed in order to simultaneously achieve three types of reaction in a single pass i.e. i) dehydrogenation of methylcyclohexane to produce toluene and hydrogen, ii) reduction of CO2 to produce active alkylating species and iii) alkylation of toluene to produce alkylated aromatics. The present invention has notable advantages over existing processes as it includes alkylation of toluene to a significant extent using CO2 or species derived from CO2 as alkylation reagent, which was not achievable in the earlier reported processes.

Recently alkylation of aromatic hydrocarbons using alkylation species derived from CO2 or CO in presence of molecular H2 is reported. Reference can be made to a Chinese patent CN104557425A, which discloses a method for production of p-xylene through aryl alkylation over a dual-function catalyst wherein alkylation reagent comprises CO or CO2 and H2 gas; aromatic hydrocarbon raw material was the miscellany of toluene, benzene and any two arbitrary proportions thereof.

Reference can be made to a US patent U.S. Pat. No. 7,902,414B, which describes a catalytic process for the selective production of p-xylene through alkylation of aromatic hydrocarbons selected from the group consisting of toluene, benzene or mixtures thereof wherein alkylation reagent comprises CO and H2. In the processes disclosed in patent CN104557425A and U.S. Pat. No. 7,902,414B, an alkylation reagent comprising CO or CO2 and molecular H2 is used to carry out alkylation of aromatic hydrocarbons. However, the present invention differs from this prior art in terms of source of H2 used. In the present invention, methylcyclohexane is used as source of H2 instead of using molecular H2.

Reference can be made to the Journal “Sci. Adv. 2020; 6: 5433”, wherein a catalytic process for the selective methylation of toluene using CO2 and molecular H2 to produce p-xylene over a ZnZrOx-HZSM-5 dual-functional catalyst was disclosed.

Reference can be made to the Journal “Chem. Lett. 2022, 51, 149-152”, wherein catalytic methylation of benzene using CO2 and molecular H2 over combinations of supported metal catalysts and zeolites is disclosed.

Reference can be made to the Journal “ChemCatChem 2020, 12, 2215-2220”, wherein methylation of benzene using CO2 and molecular H2 in the presence of catalyst comprising TiO2-supported Re and Mordenite (SiO2/Al₂O₃=90) is described.

The above-mentioned processes deal with alkylation of aromatic hydrocarbons using CO2 and molecular H2, which approach lacks the merits of the present invention wherein methylcyclohexane is used as an in-situ source of H2 instead of using molecular H2. Moreover, in the present invention methylcyclohexane serves as a source of toluene, which becomes an in-situ substrate for the required alkylation reactions

Alkylation of aromatics is reported using various alkylation reagents which include methanol, dimethylether, methylchloride, methylbromide, methylcarbonate, acetaldehyde, dimethoxyethane, acetone, olefins, dimethylsulfide, etc.

Reference can be made to a US patent U.S. Pat. No. 7,396,967B2, wherein a process for methylation of benzene and toluene to produce alkylated products, preferentially xylene is disclosed. Methylation of benzene, toluene or mixture thereof is conducted with methylating agent under vapor phase condition in presence of HZSM-5 based catalyst. Wherein, methylating agent was selected from the group consisting of methanol, dimethylether, methylchloride, methylbromide, methylcarbonate, acetaldehyde, dimethoxyethane, acetone, and dimethylsulfide.

Reference can be made to a US patent U.S. Pat. No. 9,469,579 B2, which discloses a process for producing p-xylene through the methylation of toluene using methanol as a methylating agent in presence of a catalyst comprising HZSM-5 which has been activated with a silicon compound.

Reference can be made to a patent CN102372582A, which discloses a catalytic method for toluene methylation using methanol as methylating reagent.

Reference can be made to a patent CN102372585A, which discloses a method for alkylation of aromatics to produce p-xylene using methanol or dimethylether as alkylation reagent.

Reference can be made to a patent CN102875321A, which discloses a method for continuous production of p-xylene through aromatic hydrocarbon alkylation using methanol as alkylation reagent.

Reference can be made to a patent CN102372589B, which relates to a moving bed catalytic process for preparing p-xylene by alkylating aromatic hydrocarbon using dimethylether as alkylation reagent.

Reference can be made to a patent CN102372583B, which relates to a fluidized catalytic method for preparing p-xylene by alkylating toluene using methanol and/or dimethylether as alkylation reagent.

Reference can be made to a patent CN102372586B, which discloses a fluidized catalytic method for the production of p-xylene by methylation of aromatic hydrocarbon using dimethylether as alkylation reagent

Reference can be made to a patent CN102875319A, which relates to a moving bed catalytic method of aromatics methylation using methanol as alkylation reagent.

Reference can be made to a patent CN102372584B, which relates to a fluidized catalytic method for the production of p-xylene by alkylating aromatic hydrocarbon using methanol/dimethylether as alkylation reagent.

Reference can be made to a patent U.S. Pat. No. 4,158,024A, which discloses a catalytic process for the selective production of p-xylene by contacting toluene with a methylating agent in the presence of a catalyst comprising a crystalline aluminosilicate zeolite which has combined therewith magnesium in an amount of at least about 0.5 percent by weight. Methanol, methylchloride, methylbromide, dimethylether, methyl carbonate, light olefins, or dimethylsulfide were used as methylating agent.

In the above-mentioned processes for alkylation of aromatic hydrocarbons either of the alkylation reagents such as methanol, dimethylether, methylchloride, methylbromide, methylcarbonate, acetaldehyde, dimethoxyethane, acetone, dimethylsulfide, olefins, etc., is used. These processes are entirely different from the present invention as the present invention deals with the alkylation of aromatic hydrocarbons using CO₂ or species derived from CO₂ as alkylation reagent and methylcyclohexane as H₂ donor instead of using conventional alkylation reagents.

### Objects of Aspects of the Invention

The object of one aspect of the present invention is to provide a single-step catalytic process for the production of alkylated aromatics using CO2 as a carbon source & alkylation reagent and methylcyclohexane as a hydrogen atom donor as well as source of toluene, a key intermediate for aromatics alkylation.

Another object of an aspect of the present invention is to utilize methylcyclohexane as a model H2-donor for CO2 hydrogenation to form alkylating species in the process of aromatics alkylation.

Yet another object of an aspect of the invention is to provide metal functionalized zeolite-based catalyst possessing dehydrogenation, hydrogenation and acid functionalities for the alkylation of aromatic hydrocarbons.

### BRIEF DESCRIPTION OF DRAWING

FIG. 1 describes the yields of alkylated aromatics obtained over 1 Pt-2.5Cr-2.5Zn/HZSM-5 and 1 Pt-5Cr-1 Mg/HZSM-5 catalysts.

FIG. 2 illustrates various steps involved in the production of alkylated aromatics using CO₂ as alkylation reagent and methylcyclohexane as H₂-donor and source of toluene.

### SUMMARY

Accordingly, the present invention provides a single-step catalytic process for the production of alkylated aromatics using CO2 as a carbon source & alkylation reagent and methylcyclohexane as a hydrogen atom donor as well as source of toluene over metal functionalized zeolite-based catalyst possessing active site for dehydrogenation, hydrogenation and alkylation reaction.

Present invention provides a catalytic process for the production of alkylated aromatics using CO2 as a carbon source & alkylation reagent and methylcyclohexane as a hydrogen atom donor as well as source of toluene comprising the steps of:
loading metal functionalized zeolite [HZSM-5] based catalyst into a fixed-bed micro-reactor followed by reducing the catalyst under the continuous flow of H2 gas at temperature in the range of 450 to 500° C. for a period in the range of 5 to 6 h to obtain reduced catalyst;
passing methylcyclohexane with flow rate of 2-3 WHSV (weight hourly space velocity) and mixture of CO2 and N2 (CO2/N2=1/3 vol/vol) over the reduced catalyst as obtained in step (a), at reaction temperature ranging between 300 to 400° C. and pressure ranging between 20-30 bar to produce toluene and H2;
reacting CO2 with H2 as obtained in step (b) in the presence of catalyst under the condition effective to produce active alkylating species through the hydrogenation of CO2;
reacting toluene as obtained in step (b) with alkylating species as obtained in step (c) in presence of catalyst under the condition effective for alkylation of toluene to produce alkylated aromatics.

In yet another embodiment, present invention provides a yield of the total aromatic hydrocarbons and alkylated aromatics is in the range of 25.7 to 28.6 wt % and 19.5 to 21 wt % respectively.

In yet another embodiment, present invention provides a yield of the mix xylenes, toluene and benzene is in the range of 9 to 9.5 wt %; 5.6 to 6.1 wt % and 0.6 of 1.5 wt % respectively.

In yet another embodiment of the present invention, all above mentioned steps are carried out over single multifunctional catalyst in a single pass. In yet another embodiment of the present invention, CO2 or hydrogenated species thereof is used as an alkylation reagent.

The present invention provides a metal functionalized zeolite [HZSM-5] based catalyst wherein mole ratio of silica/alumina in HZSM-5 zeolite is ranging between 30-80; surface area is ranging between 405-425 m2/g; and metals are Pt and one or more from 4th period d-block transition metals. In an embodiment of the present invention, 4th period d-block transition metals are selected from Cr, Zn or combination thereof.

In another embodiment of the present invention, metal functionalized zeolite-based catalysts are optionally promoted with alkaline earth metal and the most preferred alkaline earth metal is Mg.

In yet another embodiment, present invention provides a process for the preparation of the metal functionalized zeolite [HZSM-5] based catalyst comprising the steps of:
dissolving metal precursors in distilled water to prepare metal precursor solution;
mixing the metal precursor solution as obtained in step (i) in HZSM-5 drop-wise with continuous mixing to obtain HZSM-5 mixed metal precursor solution;
drying the HZSM-5 mixed metal precursor solution as obtained in step (ii) at temperature in the range of 100 to 110° C. for a period in the range of 10 to 12 h followed by calcination at a temperature in the range of 450 to 500° C. in muffle furnace for a period in the range of 4 to 5 h to obtain the metal functionalized zeolite [HZSM-5] based catalyst.

In yet another embodiment of the present invention, metal precursors are hexachloroplatinic acid hexahydrate, chromium nitrate nonahydrate, zinc nitrate hexahydrate and magnesium nitrate hexahydrate for Pt, Cr, Zn and Mg respectively. In yet another embodiment of the present invention, HZSM-5 used is in the form of extrudates with diameter ranging between 1.2 to 1.5 mm and length ranging between 5-10 mm. In yet another embodiment of the present invention, catalysts are optionally promoted with alkaline earth metal and most preferred alkaline earth metal is Mg.

### DETAILED DESCRIPTION

The present invention describes a single-step catalytic process for the production of alkylated aromatics using CO2 as a carbon source and alkylation reagent and methylcyclohexane as a hydrogen atom donor as well as source of toluene over metal functionalized HZSM-5 catalyst possessing dehydrogenation, hydrogenation and acid functionalities. The catalyst possesses acid sites originated from HZSM-5 and dehydrogenation and hydrogenation sites originated from metals e.g. Pt, Cr, Zn, Mg or combination thereof.

The metal functionalized HZSM-5 catalyst facilitates dehydrogenation of methylcyclohexane to produce toluene and hydrogen, hydrogenation of CO2 to form active alkylation species, reaction of alkylation species with toluene to produce xylene-rich alkylated aromatic products.

The present invention provides a process for the preparation of metal functionalized HZSM-5 catalysts by co-impregnation of metals on HZSM-5, wherein said process comprising the steps of: drying HZSM-5 at 110° C. for 12 h; wherein HZSM-5 in the form of extrudates with diameter of 1.5 mm and length of 5-10 mm; preparing metal precursor solution by dissolving required amount of the metal salts in distilled water wherein metals are Pt and one or more from 4th period d-block transition metals with or without alkaline earth metal; mixing the metal precursor solution and HZSM-5 extrudates by drop-wise addition of the solution with continuous mixing to ensure uniform impregnation of the metals on HZSM-5 surface; drying the HZSM-5 extrudates mixed with metal precursor solution at 110° C. for 12 h followed by calcinations at 500° C. for 5 h to obtain the metal functionalized HZSM-5 catalysts.

The most preferred 4th period d-block transition metals are Cr, Zn or combination thereof and most preferred alkaline earth metal is Mg.

The present invention provides a process for the preparation of Pt—Cr—Zn functionalized HZSM-5 catalyst comprising the steps of co-impregnating 1 wt % Pt, 2.5 wt % Cr and 2.5 wt % Zn on the HZSM-5 extrudates by drop wise addition of metal precursor solution prepared by dissolving required amount of hexachloroplatinic acid hexahydrate, chromium nitrate nonahydrate, zinc nitrate hexahydrate in distilled water (equivalent of water retention volume of HZSM-5), followed by drying at 110° C. for 12 h and calcinations at 500° C. for 5 h.

The present invention provides a process for the preparation of Pt—Cr—Mg functionalized HZSM-5 catalyst comprising the steps of: co-impregnating 1 wt % Pt, 5 wt % Cr and 1 wt % Mg on the HZSM-5 extrudates by drop wise addition of metal precursor solution prepared by dissolving required amount of hexachloroplatinic acid hexahydrate, chromium nitrate nonahydrate, magnesium nitrate hexahydrate in distilled water (equivalent of water retention volume of HZSM-5), followed by drying at 110° C. for 12 hour (h) and calcinations at 500° C. for 5 h.

The present invention also provides a single-step catalytic process for the production of alkylated aromatics using CO2 as a carbon source as well as alkylation reagent and methylcyclohexane as H2-donor, wherein methylcyclohexane also serves as a source of toluene.

The process is conducted in the presence of metal functionalized HZSM-5 catalyst possessing dehydrogenation, hydrogenation and acid functionalities. The catalytic process comprising the steps of: loading of the metal functionalized HZSM-5 catalyst in the fixed-bed micro-reactor and reducing the catalyst at 500° C. for 5 h in the flow of H2 gas; passing methylcyclohexane over the reduced catalyst at the feed flow rate of 2-3 WHSV, simultaneously, passing mixture of CO2 and N2 (CO2/N2=1/3 vol/vol), at the reaction temperature of 300-400° C. and 20-30 bar pressure; collecting products and qualitative-quantitative analysis of the products using gas chromatograph.

In the present invention, alkylated aromatics yield in the range of 19.5 to 21 wt % with 9 to 9.5 wt % yield for mix xylenes could be achieved over the metal functionalized HZSM-5 catalysts. Concentration of benzene in the product is very low (up to 1.5 wt %) indicating the extent of disproportionation reaction of toluene is minimum and most of the alkylated aromatics are formed through alkylation reaction.

The single-step catalytic process for the production of alkylated aromatics using CO2 as a carbon source & alkylation reagent and methylcyclohexane as a hydrogen atom donor as well as source of toluene comprises the sequential steps of; providing feedstocks such as methylcyclohexane as source of H2 and toluene; providing feedstock flow rate of 2-3 WHSV (weight hourly space velocity); providing mixture of CO2 and N2 (CO2/N2=1/3 vol/vol) in the flow rate of 160 ml/min; providing reaction temperature of 300-400° C.; providing reaction pressure of 20-30 bar; cooling of the product to obtain liquid product and gaseous product followed by their analysis using gas chromatograph.

The present invention describes development of a novel, single-step catalytic process for the production of xylene-rich alkylated aromatics which have many industrial applications e.g. in polyester industry, printing, rubber, leather processing, as a component of lubricants in motor oil, paints, polishes, adhesives, antifreeze, solvents for cleaning and degreasing, fuel additive, etc.

### EXAMPLES

Following examples are given by way of illustration of the present invention and therefore should not be construed to limit the scope of the present invention.

### Example 1

This example illustrates the preparation of Pt—Cr—Zn functionalized HZSM-5 zeolite catalyst by incipient wetness impregnation method wherein the amount of metals in the catalyst is Pt-1 weight %, Cr-2.5 weight % (wt. %) and Zn-2.5 wt. %. In typical catalyst preparation procedure, metal precursor solution was prepared by dissolving respective metal salts equivalent to 1 wt. % Pt, 2.5 wt. % Cr, 2.5 wt. % Zn into the 5 ml of distilled water (equivalent of water retention volume of HZSM-5), wherein hexachloroplatinic acid hexahydrate (0.28 gram (g)), chromium nitrate nonahydrate (2.05 g), zinc nitrate hexahydrate (1.21 g) are used as a precursor for Pt, Cr and Zn respectively. This metal precursor solution was added drop wise to the 10 g of HZSM-5 extrudates, wherein HZSM-5 has silica/alumina mole ratio of 30. Continuous mixing was done during addition of the metal precursor solution to the HZSM-5 extrudates to ensure uniform metal impregnation. The ZSM-5 extrudates mixed with metal precursor solution were kept undisturbed at room temperature for 6 h followed by drying at 110° C. for 12 h and calcinations at 500° C. for 5 h. The catalyst obtained is denoted as 1 Pt-2.5Cr-2.5Zn/HZSM-5.

### Example 2

This example illustrates the preparation of Pt—Cr—Mg functionalized HZSM-5 zeolite catalyst by incipient wetness impregnation method wherein the amount of metals in the catalyst is Pt-1 weight %, Cr-5 weight % and Mg-1 weight %. In typical catalyst preparation procedure, metal precursor solution was prepared by dissolving respective metal salts equivalent to 1 wt. % Pt, 5 wt. % Cr, 1 wt. % Mg into the 5 ml of distilled water (equivalent of water retention volume of HZSM-5), wherein hexachloroplatinic acid hexahydrate (0.28 g), chromium nitrate nonahydrate (4.14 g), magnesium nitrate hexahydrate (1.13 g) are used as a precursor for Pt, Cr and Mg respectively. This metal precursor solution was added drop wise to the 10 g of HZSM-5 extrudates, wherein HZSM-5 has silica/alumina mole ratio of 30. Continuous mixing was done during addition of the metal precursor solution to the HZSM-5 extrudates to ensure uniform metal impregnation. The ZSM-5 extrudates mixed with metal precursor solution were kept undisturbed at room temperature for 6 h followed by drying at 110° C. for 12 h and calcinations at 500° C. for 5 h. The catalyst obtained is denoted as 1 Pt-5Cr-1 Mg/HZSM-5.

### Example 3

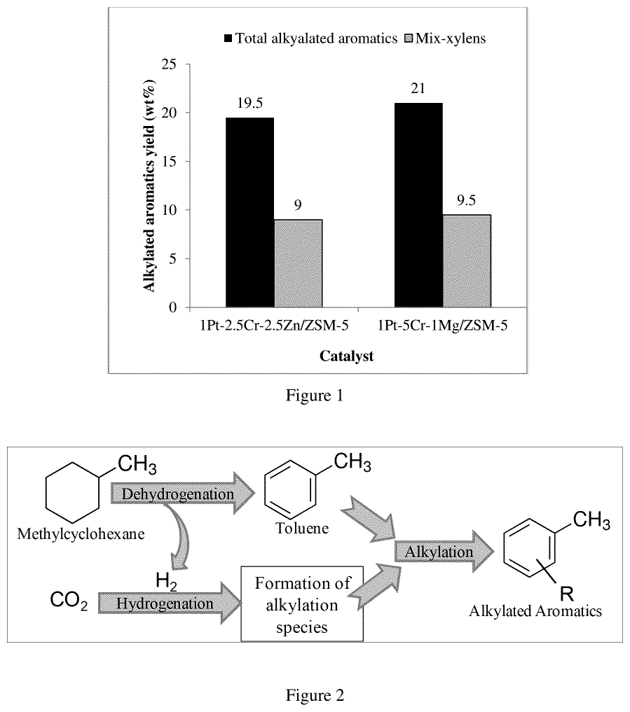

This example illustrates the performance of the 1 Pt-2.5Cr-2.5Zn/HZSM-5 catalyst (wherein the amount of metals in the catalyst is Pt-1 weight %, Cr-2.5 weight % and Zn-2.5 weight %) towards the production of alkylated aromatics using CO2 as a carbon source & alkylation reagent and methylcyclohexane as a H2-donor as well as source of toluene. In a typical run, 4 g of the 1 Pt-2.5Cr-2.5Zn/HZSM-5 catalyst was loaded in the reactor and reduced at 500° C. for 5 h in the flow of H2 gas and then the reactor was allowed to cool down to 30° C. Next, the reactor was flushed with N2 gas and then heated to 360° C. temperature in the flow of N2 gas. At 360° C., the flow of N2 gas was stopped, methylcyclohexane was pumped into the reactor with WHSV of 2. Simultaneously, mixture of CO2 and N2 (CO2/N2=1/3 vol/vol) with flow rate of 160 ml/min was supplied to the reactor. The catalytic run was carried out at 360° C. and at 30 bar pressure. The products were analyzed using gas chromatograph. The data given in table 1 (entry 1) shows that over 1 Pt-2.5Cr-2.5Zn/HZSM-5 catalyst (prepared as per the procedure said in example 1), 25.7 wt % yield of aromatics hydrocarbon was obtained. Distribution of aromatic products as per their carbon numbers is shown in table 1 (entry 1). As shown in FIG. 1, over the 1 Pt-2.5Cr-2.5Zn/HZSM-5 catalyst 19.5 wt % yield of total alkylated aromatics was obtained with 9 wt % yield for mix xylenes.

TABLE 1
Aromatic hydrocarbons yield obtained over 1Pt—2.5Cr—2.5Zn/HZSM-
5catalyst and 1Pt—5Cr—1Mg/HZSM-5 catalyst.

| | | Aromatic Hydrocarbons yield (wt %) | | | | | | |
|---|---|---|---|---|---|---|---|---|
| | | | | | | | | Total |
| Sr. | | | | | | | | aro- |
| No. | Catalyst | C₆ | C₇ | C₈ | C₉ | C₁₀ | C₁₀₊ | matics |
| 1 | 1Pt—2.5Cr—2.5Zn/ | 0.6 | 5.6 | 10.4 | 6.0 | 2.0 | 1.1 | 25.7 |
| | HZSM-5 | | | | | | | |
| 2 | 1Pt—5Cr—1Mg/ | 1.5 | 6.1 | 11.0 | 6.4 | 2.2 | 1.4 | 28.6 |
| | HZSM-5 | | | | | | | |

### Example 4

This example illustrates the performance of the 1 Pt-5Cr-1 Mg/HZSM-5 catalyst (wherein the amount of metals in the catalyst is Pt-1 weight %, Cr-5 weight % and Mg-1 weight %) towards the production of alkylated aromatics using CO2 as a carbon source & alkylation reagent and methylcyclohexane as a H2-donor as well as source of toluene. In a typical run, 4 g of thelPt-5Cr-1 Mg/HZSM-5 catalyst was loaded in the reactor and reduced at 500° C. for 5 h in the flow of H2 gas and then the reactor was allowed to cool down to 30° C. Next, the reactor was flushed with N2 gas and then heated to 360° C. temperature in the flow of N2 gas. At 360° C., the flow of N2 gas was stopped, methylcyclohexane was pumped into the reactor with WHSV of 2. Simultaneously, mixture of CO2 and N2 (CO2/N2=1/3 vol/vol) with flow rate of 160 ml/min was supplied to the reactor. The catalytic run was carried out at 360° C. and at 30 bar pressure. The products were analyzed using gas chromatograph. The data given in table 1 (entry 2) shows that over 1 Pt-5Cr-1 Mg/HZSM-5 catalyst (prepared as per the procedure said in example 2), 28.6 wt. % yield of aromatic hydrocarbons was obtained. Distribution of aromatic products as per their carbon numbers is shown in table 1 (entry 2). As shown in FIG. 1, over 1 Pt-5Cr-1 Mg/HZSM-5 catalyst 21 wt. % yield of total alkylated aromatics was obtained with 9.5 wt. % yield for mix xylenes.

### Advantages of the Invention

Certain advantages of aspects of the present invention include, but are not limited to:

The process is advantageous through environmental aspect as it utilizes CO2, a major greenhouse gas instead of chemical like methanol or dimethylether as alkylation reagent to produce valuable alkylated aromatic hydrocarbons.

Application of a liquid organic hydrogen carrier i.e. methylcyclohexane as a H2-donor instead of molecular H2 for CO2 hydrogenation is advantageous as liquid organic hydrogen carriers are easy to handle/transport than gaseous molecular H2. The concept of using liquid organic hydrogen carrier can further be extended to the inexpensive H2-donors other than methylcyclohexane.

In aspects of the present invention methylcyclohexane serves as H2-donor as well as source of toluene. The process is more atom-economical as dehydrogenated species of H2-donor (toluene) is utilized in-situ as one of the reactants during the course of reaction.

In some embodiments, the invention provides metal functionalized zeolite-based multifunctional catalyst capable of carrying out dehydrogenation of methylcyclohexane, hydrogenation of CO2 and alkylation of toluene in single pass which reduces the number of steps involved in the synthesis of alkylated aromatics.

The process also has advantage of achieving 19.5-21 wt. % yield for alkylated aromatics with 9-9.5 wt. % yield for mix xylenes from methylcyclohexane and CO2. The selectivity for mix xylenes amongst total alkylated products is >45 wt. % which highlights the industrial importance of the process for the production of xylenes.

The present invention provides an alternate proficient route for the alkylation of aromatics using CO2 which reduces net greenhouse gas emission in the process.

The process described in the present invention can be coupled with the existing processes known for methylcyclohexane production for continuous supply and further scale-up.

What is claimed is:

1. A catalytic process for production of alkylated aromatics using CO₂ and methylcyclohexane, wherein CO₂ is a carbon source and an alkylation reagent, and methylcyclohexane is a hydrogen atom donor and a source of toluene, the catalytic process comprising:
preparing a metal functionalized zeolite (HZSM-5) based catalyst;
loading the metal functionalized zeolite (HZSM-5) based catalyst into a fixed-bed micro-reactor followed by reducing the metal functionalized zeolite (HZSM-5) based catalyst under a continuous flow of H2 gas at a temperature in a range of 450 to 500° C. for a period in a range of 5 to 6 h to obtain a reduced catalyst;
passing methylcyclohexane and a mixture of CO₂ and N₂ (CO₂/N₂=1/3 vol/vol) over the reduced catalyst, wherein methylcyclohexane is passed at a flow rate of 2-3 WHSV (weight hourly space velocity) at a reaction temperature ranging between 300 to 400° C. and a pressure ranging between 20-30 bar to produce toluene and H₂;
reacting CO₂ with H₂ in presence of the reduced catalyst to produce an active alkylating species through hydrogenation of CO₂; and
reacting toluene with the alkylating species in presence of the reduced catalyst for alkylating toluene to produce aromatic hydrocarbons, and alkylated aromatics.

2. The catalytic process as claimed in claim 1, wherein yield of the aromatic hydrocarbons is in a range of 25.7 to 28.6 wt. % and yield of alkylated aromatics is in a range of 19.5 to 21 wt. %.

3. The catalytic process as claimed in claim 2, wherein the aromatic hydrocarbons comprise mix xylenes in a range of 9 to 9.5 wt. %, toluene in a range of 5.6 to 6.1 wt. % and benzene in a range of 0.6 of 1.5 wt. %.

4. The catalytic process as claimed in claim 1, wherein preparing metal functionalized zeolite (HZSM-5) based catalyst comprises:
dissolving metal precursors in distilled water to prepare a metal precursor solution;
mixing the metal precursor solution in HZSM-5 drop-wise with continuous mixing to obtain a HZSM-5 mixed metal precursor solution; and
drying the HZSM-5 mixed metal precursor solution at a temperature in a range of 100 to 110° C. for a period in a range of 10 to 12 hours followed by calcination at a temperature in a range of 450 to 500° C. in a muffle furnace for a period in a range of 4 to 5 hours to obtain the metal functionalized zeolite (HZSM-5) based catalyst.

5. The catalytic process as claimed in claim 4, wherein metal precursors are hexachloroplatinic acid hexahydrate, chromium nitrate nonahydrate, zinc nitrate hexahydrate and magnesium nitrate hexahydrate for Pt, Cr, Zn, and Mg, respectively.

6. The catalytic process as claimed in claim 4, wherein the HZSM-5 used is in a form of extrudates with a diameter ranging between 1.2 to 1.5 mm and a length ranging between 5 to 10 mm.

7. The catalytic process as claimed in claim 4, wherein the metal functionalized zeolite (HZSM-5) based catalyst is promoted with an alkaline earth metal.

8. The catalytic process as claimed in claim 7, wherein the alkaline earth metal is Mg.

9. The catalytic process as claimed in claim 1, wherein the metal functionalized zeolite (HZSM-5) based catalyst comprises HZSM-5 zeolite, and metals, wherein the HZSM-5 zeolite has a mole ratio of silica/alumina in a range between 30-80 and a surface area ranging between 405-425 m²/g, and wherein the metals are Pt and one or more from 4th period d-block transition metals.

10. The catalytic process as claimed in claim 1, wherein the methylcyclohexane is used as an in-situ source of H₂.

11. The catalytic process as claimed in claim 1, wherein the catalytic process is carried out in absence of molecular H₂.

12. The catalytic process as claimed in claim 1, wherein the catalytic process is a single step process.

13. A metal functionalized zeolite (HZSM-5) based catalyst comprising: HZSM-5 zeolite, and metals, wherein the HZSM-5 zeolite has a mole ratio of silica/alumina in a range between 30-80 and a surface area ranging between 405-425 m²/g, and wherein the metals are Pt and one or more from 4th period d-block transition metals.

14. The metal functionalized zeolite (HZSM-5) based catalyst as claimed in claim 13, wherein 4th period d-block transition metals are Cr, Zn, or a combination thereof.

15. The metal functionalized zeolite (HZSM-5) based catalyst as claimed in claim 13, wherein the metal functionalized zeolite (HZSM-5)-based catalyst is promoted with alkaline earth metal.

16. The metal functionalized zeolite (HZSM-5) based catalyst as claimed in claim 15, wherein the alkaline earth metal is Mg.

17. The metal functionalized zeolite (HZSM-5) based catalyst as claimed in claim 13, wherein the metal functionalized zeolite (HZSM-5) based catalyst possesses acid sites originated from HZSM-5 and dehydrogenation and hydrogenation sites originated from metals.

18. The metal functionalized zeolite (HZSM-5) based catalyst as claimed in claim 17, wherein the metals are Pt, Cr, Zn, Mg, or a combination thereof.

19. The metal functionalized zeolite (HZSM-5) based catalyst as claimed in claim 13, wherein the metal functionalized zeolite (HZSM-5) based catalyst is 1 Pt-2.5Cr-2.5Zn/HZSM-5, or 1 Pt-5Cr-1 Mg/HZSM-5.
